(19) United States
(12) Patent Application Publication
(10) Pub. No.: US 2023/0371840 A1
(43) Pub. Date: Nov. 23, 2023

(54) IMAGING PLUG-IN DEVICE

(71) Applicant: Sino Canada Health Engineering Research Institute (Hefei) Ltd., Anhui (CN)

(72) Inventors: Haoqin Zhu, Anhui (CN); Jin Su, Anhui (CN); Gong Zhang, Anhui (CN)

(21) Appl. No.: 18/246,294

(22) PCT Filed: Sep. 27, 2020
(86) PCT No.: PCT/CN2020/118048

§ 371 Date: Mar. 22, 2023

(51) Int. Cl. A61B 5/055; A61B 6/00

(52) U.S. Cl. CPC A61B 5/055; A61B 6/4452

(57) ABSTRACT

Provided is an MR/PET integrated imaging plug-in device that may be inserted into an MM system without requiring modifications to the MRI system, including: a to-be-detected object holder at the magnetic field generated by the magnetic field generation structure, and configured to carry the to-be-detected object; a PET detection component configured to detect a PET image signal from the to-be-detected object; a magnetic resonance receiving coil configured to generate a magnetic resonance image of the to-be-detected object a signal amplification component. The PET detection component is movable relative to the to-be-detected object holder to align or leave the to-be-detected object. The MR/PET integrated imaging plug-in device combines PET and MRI medical imaging modes into one device, so that people may use PET to obtain high-sensitivity images of in vivo molecular processes, and use MRI to obtain high-quality anatomical information, while overcoming the limitation of high cost of whole-body synchronous PET/MRI.

### TECHNICAL FIELD

The present disclosure relates to a field of medical imaging technology, and in particular, to an imaging plug-in device.

### BACKGROUND

Positron Emission Computed Tomography (PET) has a sensitivity of up to a nanogram molecular level and may well obtain the functional and metabolic information of a to-be-detected object. However, a spatial resolution of the Positron Emission Computed Tomography is low. Magnetic Resonance Imaging (MRI) technology is widely used in clinical medicine, etc. An advantage of MRI imaging technology is high spatial resolution, which may obtain the structural information of the to-be-detected object. However, the sensitivity may only reach a milligram molecular level, and the acquisition of functional and metabolic information is not satisfactory. At present, science and clinic are developing and researching more advanced fusion images with anatomical structure and physiological and biochemical information, so as to provide information such as a development cycle from abnormal cell metabolism to structural variability, and provide more basis for clinical and scientific research of staging.

For example, a human brain may be imaged simultaneously using PET and MRI to improve imaging sensitivity and accuracy, so as to meet a user's requirements for anatomical and metabolic information. For neurodegenerative diseases, such as Alzheimer's disease and Parkinson's disease, diagnosis of brain cancer, treatment monitoring and staging, and planning and evaluation of epilepsy surgery, MRI imaging and PET imaging technology may be used together for imaging, and then image fusion may be performed to improve an imaging effect, so as to meet the requirements of relevant research and clinical application.

In order to obtain a PET image and a MRI image of a to-be-detected object for image fusion, in the related art, the PET system and the MM system may be integrated in a device to form a PET/MRI system. However, these scanners are complex and expensive, which makes many institutions unable to afford. In addition, for institutions that have purchased the MRI system which is in normal use, if the institutions further purchase the PET/MRI system, a resource waste is caused, and a new solution is required.

### SUMMARY

An objective of the present disclosure is to integrate a new PET imaging system on a MRI system to realize a PET/MRI integrated system, thus solving a problem that the PET/MRI system is expensive and causes a resource waste of the existing MRI system.

The present disclosure provides an imaging plug-in device, applied to a magnetic resonance imaging system (MRI system for short). The magnetic resonance imaging system may include: a magnet, a gradient coil, a built-in body coil, a hospital bed, a gradient power amplifier, a radio frequency power amplifier, a spectrometer, a radio frequency front-end device, a computer host, and the like. The hospital bed is configured to carry the imaging plug-in device and a human body. The imaging plug-in device includes: a to-be-detected object holder, a PET detection component, a magnetic resonance phased coil, and a signal amplification component. The to-be-detected object holder is located at the magnetic field generated by the magnetic field generation structure, and configured to carry the to-be-detected object, and the to-be-detected object is a part of the human body. The PET detection component is configured to detect a PET signal from the to-be-detected object to generate a PET image of the to-be-detected object. The magnetic resonance phased coil at least includes a magnetic resonance receiving coil for detecting a magnetic resonance signal from the to-be-detected object and further includes a local transmitting coil configured to transmit a radio frequency signal to form a radio frequency field. The magnetic resonance signal includes a signal emitted by the to-be-detected object after being excited by the radio frequency field, which is used to generate a magnetic resonance image of the to-be-detected object. The signal amplification component is arranged to be at a distance from the magnetic resonance receiving coil less than a distance threshold to at least improve a signal-to-noise ratio of the magnetic resonance signal. The PET detection component is movable relative to the to-be-detected object holder to align the to-be-detected object or leave the to-be-detected object.

According to embodiments of the present disclosure, the magnetic resonance receiving coil and the local transmitting coil are split, and the transmitting coil is divided into two ways: a first way is that the local transmitting coil is arranged on a side of the PET detection component close to the to-be-detected object, and is called the local transmitting coil, and moves together with the PET detection component to align or leave the to-be-detected object; a second way is that the transmitting coil is a built-in body coil of the magnetic resonance imaging system.

According to embodiments of the present disclosure, the local transmitting coil is coupled to a local radio frequency transmission interface end of the magnetic resonance imaging system through an external junction box.

According to embodiments of the present disclosure, the external junction box includes a radio frequency power distributor.

According to embodiments of the present disclosure, the radio frequency power distributor is configured to generate two radio frequency signals with a same amplitude and a phase difference of 90 degrees.

According to embodiments of the present disclosure, an input end of the radio frequency power distributor is coupled to the local radio frequency transmission interface end of the magnetic resonance imaging system, and two output ends of the radio frequency power distributor are respectively coupled to the local transmitting coil.

According to embodiments of the present disclosure, the signal amplification component is arranged in the external junction box to amplify the magnetic resonance signal received by the magnetic resonance receiving coil.

According to embodiments of the present disclosure, the magnetic resonance receiving coil and the local transmitting coil are integrated, and together form a single-layer transceiver common phased array coil.

According to embodiments of the present disclosure, the single-layer transceiver common phased array coil is arranged on a side of the PET detection component close to the to-be-detected object, and moves together with the PET detection component to align or leave the to-be-detected object.

According to embodiments of the present disclosure, the single-layer transceiver common phased array coil includes a plurality of channel coils; the plurality of channel coil have respective transmit/receive switches; the transmit/receive switch is configured to switch a working state of the channel coil corresponding to the transmit/receive switch, and the working state includes: a transmitting state and a receiving state.

According to embodiments of the present disclosure, the single-layer transceiver common phased array coil is coupled to the local radio frequency transmission interface end and a receiving coil receiving end of the MRI system through the external junction box.

According to embodiments of the present disclosure, the external junction box matched with the single-layer transceiver common phased array coil further includes a multi-channel radio frequency power distributor, wherein each channel of the multi-channel radio frequency power distributor has a one-to-one correspondence with each channel coil.

According to an 8-channel single-layer transceiver common phased array coil of the present disclosure, a phase difference between adjacent channel coils is 45 degrees, and phases of each channel are not repeated.

According to embodiments of the present disclosure, the multi-channel radio frequency power distributor includes a three-level power distributor and a phase shifter.

According to embodiments of the present disclosure, the external junction box includes a signal amplification component configured to amplify the magnetic resonance signal received by the transceiver common coil.

According to embodiments of the present disclosure, at least part of the magnetic resonance receiving coil is arranged on the to-be-detected object holder; and/or at least part of the magnetic resonance receiving coil is detachably arranged on the to-be-detected object; and/or the magnetic resonance receiving coil includes a flexible phased array coil, and at least part of the flexible phased array coil is a wearable structure.

According to embodiments of the present disclosure, the PET detection component further includes a roller-like structure and a moving structure, wherein the moving structure is configured to drive the roller-like structure to move along an extension direction of the to-be-detected object holder.

According to embodiments of the present disclosure, the imaging plug-in device may be connected into the MRI system as a plug-in device, and the magnetic resonance receiving coil is used to detect the magnetic resonance signal from the to-be-detected object. The PET detection component is used to detect the PET signal from the to-be-detected object. In this way, organizations with the MRI system may obtain PET image information and MRI image information of the to-be-detected object simultaneously or in batches based on the existing MM system and the imaging plug-in device without purchasing the integrated PET/MRI system. Compared with the separate detection of to-be-detected object by the separate MRI system and PET system, the PET image information and MRI image information collected in embodiments of the present disclosure have better simultaneity and directionality, which is convenient for obtaining high-quality images after fusion.

### BRIEF DESCRIPTION OF THE DRAWINGS

The above and other objectives, features and advantages of embodiments of the present disclosure will become easier to understand through the following description with reference to the accompanying drawings. In the accompanying drawings, a plurality of embodiments of the present disclosure will be described in an exemplary and non-limiting manner.

FIG. 1 schematically shows a schematic diagram of an imaging plug-in device when performing detection according to embodiments of the present disclosure.

FIG. 2 schematically shows a schematic structural diagram of an imaging plug-in device according to embodiments of the present disclosure.

FIG. 3 schematically shows a schematic structural diagram of a PET detection component according to embodiments of the present disclosure.

FIG. 4 schematically shows a schematic structural diagram of a local transmitting coil according to embodiments of the present disclosure.

FIG. 5 schematically shows a position of a magnetic resonance receiving coil in a PET detection component according to embodiments of the present disclosure.

FIG. 6 schematically shows a schematic structural diagram of a signal amplification component according to embodiments of the present disclosure.

FIG. 7 schematically shows a schematic diagram of a single-layer transceiver common phased array coil according to embodiments of the present disclosure.

FIG. 8 schematically shows a schematic diagram of an integrated circuit of components of a single-layer transceiver common phased array coil according to embodiments of the present disclosure.

FIG. 9 schematically shows a circuit diagram of a radio frequency power distributor applicable to an eight-channel single-layer transceiver common phased array coil according to embodiments of the present disclosure.

FIG. 10 schematically shows a schematic structural diagram of a radio frequency signal generation circuit according to embodiments of the present disclosure.

FIG. 11 schematically shows a schematic diagram of local transmitting coil and junction box circuit integration according to embodiments of the present disclosure.

FIG. 12 schematically shows a schematic structural diagram of a to-be-detected object holder according to another embodiment of the present disclosure.

FIG. 13 schematically shows a structural perspective view of an imaging plug-in device according to embodiments of the present disclosure.

FIG. 14 schematically shows a front view of an imaging plug-in device according to embodiments of the present disclosure.

FIG. 15 schematically shows A-A sectional view of the imaging plug-in device in FIG. 14.

### REFERENCE SIGN

10. Imaging plug-in device; 11. PET detection component; 113. External junction box; 12. Magnetic resonance receiving coil; 14. Local transmitting coil; 15. Single-layer transceiver common phased array coil; 16. To-be-detected object holder; 17. Channel coil; P. Channel; 32. Radio frequency power distributor;
20. MRI system; 22. Hospital bed;
13. Signal amplification component; 131. Four-port directional coupling circuit; 101. Power distributor; 102. Phase shifter; 1021 and 1022. Phase lag phase shifter; 1023. Phase lead phase shifter; 132. Preamplifier; 18. Transmit/receive switch; TX. Local input radio frequency power.

### DETAILED DESCRIPTION OF EMBODIMENTS

Embodiments of the present disclosure are described in detail below. Examples of embodiments are shown in the accompanying drawings, wherein the same or similar reference signs throughout represent the same or similar elements or elements with the same or similar functions. Embodiments described below with reference to the accompanying drawings are exemplary and are intended to explain the present disclosure, but may not be construed as limiting the present disclosure.

In the present disclosure, unless otherwise explicitly stated or limited, terms “installed”, “linked”, “connected”, “secured”, and the like should be interpreted in a broad sense, e.g., it may be fixedly connected, detachably connected, or integrated; may be mechanically connected, may be electrically connected or may be in communication with each other; may be directly connected or indirectly connected through an intervening media, or may be internal communication of two elements or an interaction relationship between two elements unless otherwise specifically defined. Those skilled in the art may understand the specific meanings of the above-mentioned terms in the present disclosure according to specific situations.

In the description of the specification, the description referring to terms “one embodiment”, “some embodiments”, “examples”, “specific examples”, or “some examples” means that specific features, structures, materials, or features described in combination with the embodiment or example are included in at least one embodiment or example of the present disclosure. In the specification, the schematic expressions of the above-mentioned terms are not necessarily directed to the same embodiment or example. Furthermore, the described specific features, structures, materials or characteristics may be combined in an appropriate manner in any one or more embodiments or examples. In addition, without contradiction, those skilled in the art may incorporate and combine different embodiments or examples and features of different embodiments or examples described in the specification.

Unless otherwise defined, all technical and scientific terms used herein have the same meaning as commonly understood by those of ordinary skill in the art to which the present disclosure belongs. Although any method and material similar or equivalent to those methods and materials described herein may be used in the practice or test of the present disclosure, preferred methods and materials are now described.

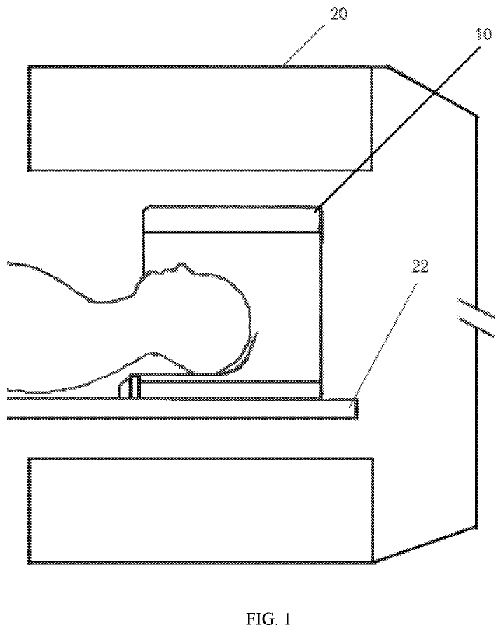

FIG. 1 schematically shows a schematic diagram of an imaging plug-in device when performing detection according to embodiments of the present disclosure.

As shown in FIG. 1, when a PET detection is performed on a to-be-detected object, at least part of an imaging plug-in device 10 may be arranged around the to-be-detected object, and as shown in FIG. 1, may be arranged around a head of a human body to collect PET data. When it is necessary to perform a MRI detection on the to-be-detected object, different modes may be used for detection. For example, the PET detection component of the imaging plug-in device 10 may be moved away from the to-be-detected object, and a local transmitting coil and a magnetic resonance receiving coil (such as a magnetic resonance receiving coil worn by a user on the head) integrated in the PET detection component 11 may be used for the MM detection.

In order to facilitate the understanding of embodiments of the present disclosure, a magnetic field generation structure is exemplarily described. The magnetic field generation structure includes but is not limited to a superconducting magnet, a resistance magnet and a permanent magnet.

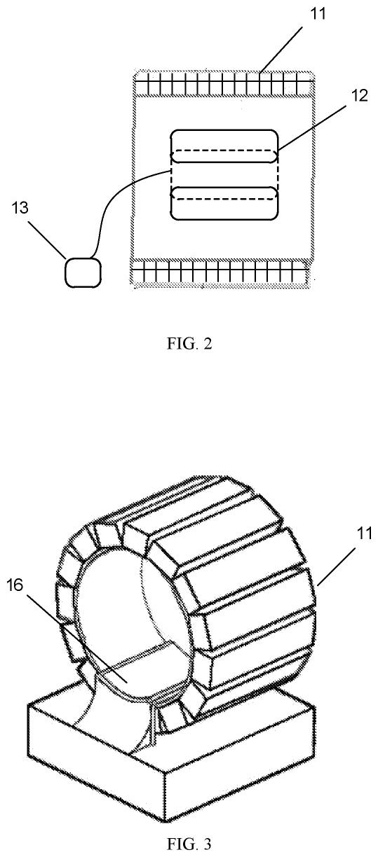

FIG. 2 schematically shows a schematic structural diagram of an imaging plug-in device according to embodiments of the present disclosure.

As shown in FIG. 2, the imaging plug-in device 10 includes: a PET detection component 11, a magnetic resonance phased coil 12 and a signal amplification component 13.

The PET detection component 11 is exemplarily described below.

FIG. 3 schematically shows a schematic structural diagram of a PET detection component according to embodiments of the present disclosure.

As shown in FIG. 3, the PET detection component 11 is used to detect a PET signal from the to-be-detected object to generate a PET image of the to-be-detected object. The PET detection component 11 is movable relative to a to-be-detected object holder 16 to align the to-be-detected object or leave the to-be-detected object, so that the to-be-detected object is located inside or outside a scanning region of the PET detection component 11.

The PET detection component 11 may have a plurality of PET detectors arranged in a preset arrangement. The arrangement may be determined according to a mechanical structure design, such as to facilitate alignment with the to-be-detected object. In addition, it is also necessary to consider the feeling of the to-be-detected object. For example, when the to-be-detected object is a user's head, the PET detection component 11 should not be too close to the user's face. For example, a plurality of PET detectors are arranged adjacent to each other in a form of an array, and formed into a ring arranged around a part of a patient's body. A size of the ring is suitable for being received in a tunnel structure of the MRI system together with the to-be-detected object. For example, the PET detector includes a plurality of side walls stacked together to form a cylinder. Silicon photomultipliers (SiPMs) on the PET detector are compatible with the MM system to generate a PET image based on a signal output by the SiPMs.

The magnetic resonance phased coil is exemplarily described below.

Firstly, the magnetic resonance phased coil at least includes a magnetic resonance receiving coil 12. During the MM detection, the PET detection component 11 leaves the to-be-detected object, so that a detected part is completely exposed to a radio frequency radiation range of a built-in body coil of a system. The magnetic resonance receiving coil 12 is used together with the built-in body coil of the Mill system to obtain a magnetic resonance image signal. Then the PET detection component may be moved back and aligned with the detected part for PET image detection. In this way, the magnetic resonance image and the PET image are collected separately. Secondly, the magnetic resonance phased coil includes a local transmitting coil 14 placed on an inner side the PET detection component, which is used together with the magnetic resonance receiving coil 12. In this way, the magnetic resonance image and the PET image are collected simultaneously. Thirdly, the magnetic resonance phased coil includes a single-layer transceiver common phased array coil fixed on an inner side of the PET detection component to obtain a magnetic resonance image. In this way, the magnetic resonance image and the PET image are collected simultaneously.

The magnetic resonance receiving coil is exemplarily described below.

In an embodiment, at least part of the magnetic resonance receiving coil 12 may be fixed on the to-be-detected object. For example, the magnetic resonance receiving coil 12 includes a wearable structure. The wearable structure includes but is not limited to a structure which is made of an elastic material and may fix the magnetic resonance receiving coil 12 on the to-be-detected object, such as an elastic band, a clamp, a velcro, etc. For example, the magnetic resonance receiving coil 12 may switch between an open loop state and a closed loop state, which is convenient for wearing.

In an embodiment, the magnetic resonance receiving coil 12 includes a flexible phased array coil. The phased array system may include 4 to 8 coils or more coils.

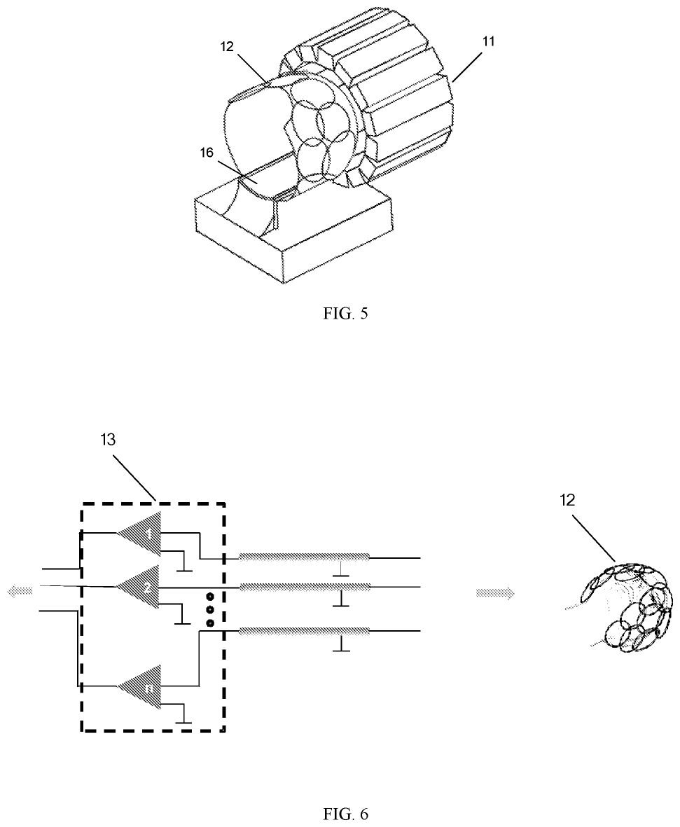

As shown in FIG. 5 and FIG. 6, the magnetic resonance phased coil includes a plurality of adjacent and at least partially overlapped circular magnetic resonance receiving coils 12. Adjacent coils overlap to minimize coupling between the adjacent coils. A shape of a single coil includes but is not limited to at least one of a polygon, a circle, an ellipse and an irregular shape.

The local transmitting coil is exemplarily described below.

In an embodiment, the magnetic resonance phased coil further includes a magnetic resonance transmitting coil for transmitting a radio frequency signal to form a radio frequency field. The magnetic resonance transmitting coil may be a body coil in the MRI system or a local transmitting coil arranged in the PET detection component 11.

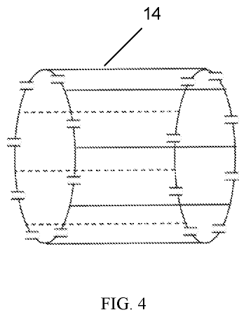

FIG. 4 schematically shows a schematic structural diagram of a local transmitting coil according to embodiments of the present disclosure.

In an embodiment, since the PET detection component 11 has a radio frequency shielding effect on the built-in body coil of the magnetic resonance system, after the PET detection component 11 is removed from the to-be-detected object, the MRI image detection is performed. The MM signal detection method is very suitable for the MRI system without a local radio frequency transmission port. For the MRI system with the local radio frequency transmission port, the MM detection may be performed on the to-be-detected object without removing the PET detection component 11. The following settings may be adopted: the local transmitting coil (hereinafter referred to as Tx coil) and the magnetic resonance receiving coil (hereinafter referred to as Rx coil) may be set in two-layer local coil, and the local transmitting coil 14 and the magnetic resonance receiving coil 12 are installed in the PET detection component 11, wherein the two-layer coil is composed of the local transmitting coil 14 (such as a local birdcage transmitting coil) located in an outer layer and the magnetic resonance receiving coil 12 (such as a phased array receiving coil) located in an inner layer.

In an embodiment, since a certain distance is required to be maintained between the Tx coil and the Rx coil in order to eliminate an mutual interference between the Tx coil and the Rx coil, a mechanical design complexity increases when both the Tx coil and the Rx coil are fixed in the PET detection assembly 11. In addition, the PET detector module 11 is large in size due to a large space required for the Tx coil, the Rx coil, an electronic component, and the like, and is not easily disposed in a tunnel-shaped detection space of the MM. In addition, a space available for accommodating the to-be-detected object in PET detection component 11 is also squeezed. When the space available for accommodating the to-be-detected object is too small, a user experience will be reduced. In order to solve the above-mentioned problem, a single-layer transceiver common phased array coil mode may be adopted.

In an embodiment, the magnetic resonance receiving coil 12 and the magnetic resonance transmitting coil 14 are integrated to form the single-layer transceiver common phased array coil 15, so that the MRI detection may be realized through the single-layer transceiver common phased array coil 15.

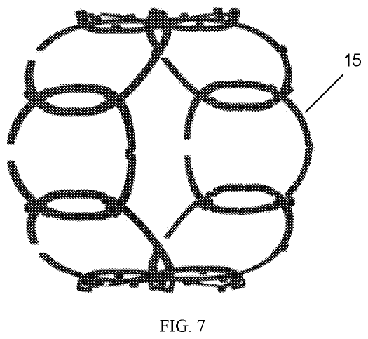

FIG. 7 schematically shows a schematic diagram of a single-layer transceiver common phased array coil according to embodiments of the present disclosure. The single-layer transceiver common phased array coil 15 is arranged at a side of the PET detection component 11 close to the to-be-detected object, and moves together with the PET detection component 11. Alternatively, the single-layer transceiver common phased array coil 15 may be arranged separately with PET detection component 11, for example, fixed on the to-be-detected object holder 16.

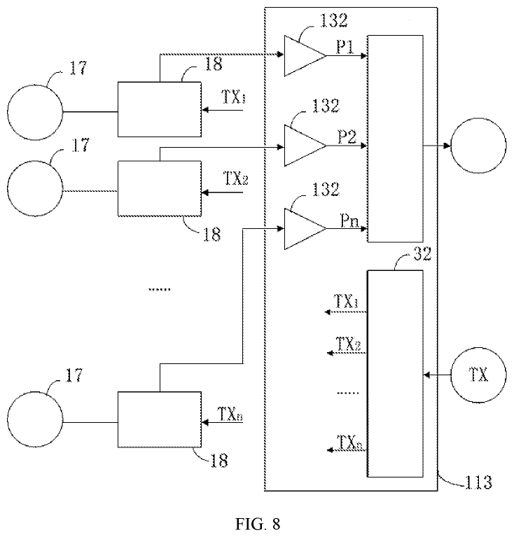

FIG. 8 schematically shows a schematic diagram of an integrated circuit of components of a single-layer transceiver common phased array coil according to embodiments of the present disclosure.

The imaging plug-in device 10 further includes a multi-channel radio frequency power distributor, and the multi-channel radio frequency power distributor includes an input end and a plurality of output ends. The number of output ends is the same as the number of channels of the phased array coil. The amplitude of the output signal at each output end is the same, and the phase difference corresponds to the phase difference between each channel coil 17. The input end is connected with the local radio frequency transmission interface end of the MRI system. The output end is connected with the corresponding channel P of the phased array coil through a transmit/receive switch (T/R switch) 18. At the same time, each channel of the phased array coil is connected with a corresponding preamplifier 132. In the embodiment, the transceiver common magnetic resonance phased array coil is characterized by single-layer coil and space saving.

As shown in FIG. 8, a radio frequency power distributor 32 allocates a radio frequency power (TX1 to TXn) to each channel by local input radio frequency power TX. The transmit/receive switch 1 to the transmit/receive switch n are used to switch a working state of channel coil 1 to channel coil n.

In an embodiment, an 8-channel coil is used as an example for illustration.

As shown in FIG. 9, each power distributor 101 may be a dual-channel 90-degree phase difference radio frequency power distributor (Hybrid 90°), and the phase shifter 102 may include a 45° phase lag phase shifter 1021, a 90° phase lag phase shifter 1022, and a 45° phase lead phase shifter 1023. The phase difference between adjacent channels may be 45 degrees, such as 0°, −45°, −90°, −135°, −180°, −225°, −270°, and −315°. A reference point is that a phase of a local radio frequency input end is 0°. An isolation port of each dual-channel 90-degree phase difference radio frequency power distributor needs to be grounded through a terminal resistor with a resistance value of 50 ohms.

The signal amplification component is exemplarily described below.

The signal amplification component is arranged to be at a distance from the magnetic resonance receiving coil less than a distance threshold. For example, the signal amplification component is arranged at a nearest settable place outside a region of interest of the PET detection component to obtain the best signal-to-noise ratio of the magnetic resonance signal. For example, the signal amplification component may be arranged on the imaging plug-in device in a front-end mode, such as arranged on a base accessory of the PET detection component.

FIG. 6 schematically shows a schematic structural diagram of a signal amplification component according to embodiments of the present disclosure. Each receiving coil 12 in the magnetic resonance phased coil is respectively connected with a corresponding preamplifier in the signal amplification component, and an output end of the preamplifier is connected with a receiving channel of the MRI system.

An external junction box will be described below.

The external junction box at least includes a preamplifier and a power distributor or multi-channel power distributor with a phase difference of 90 degrees, and the phase of each channel is the same as the phase of the coil in the corresponding phased array coil.

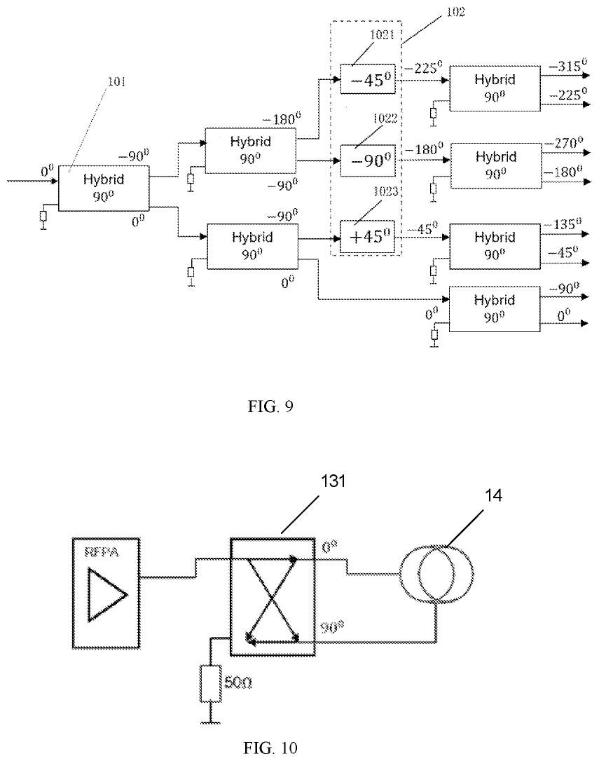

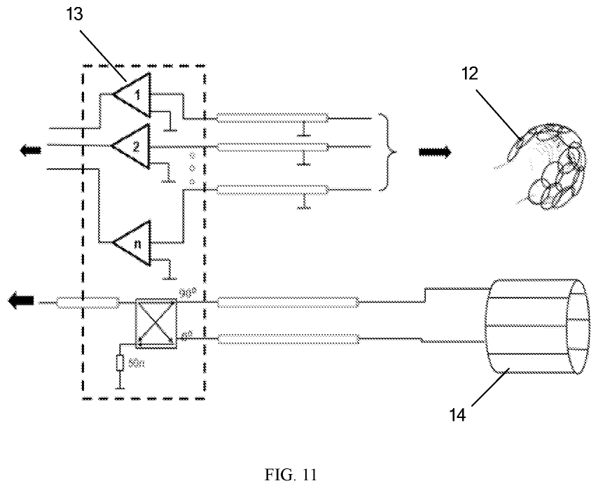

FIG. 10 shows a schematic structural diagram of a radio frequency signal generation circuit for a local birdcage transmitting coil. Referring to FIG. 11, the radio frequency generation circuit is a four-terminal network radio frequency device, which equally divides an input radio frequency power signal (RFPA) into two power output signals (such as −3 dB power divider) with a phase difference of 90 degrees.

In an embodiment, the signal amplification component is arranged in the external junction box 113, which may facilitate mode switching. When the local transmitting coil 14 is arranged in the PET detection component 11, the external junction box 113 may provide two interfaces: a signal amplification interface and a radio frequency signal interface. For another example, when the local transmitting coil 14 is not arranged in the PET detection component 11, the external junction box 113 may provide a signal amplification interface. That is, the external junction box 113 may only be provided with an amplification circuit, or the external junction box 113 may also be provided with an amplification circuit and a radio frequency generation circuit simultaneously. The radio frequency generation circuit may be used to generate two channels of radio frequency signals with a phase difference of 90 degrees or eight channels of radio frequency signals with a phase difference of 45 degrees.

It should be emphasized that the external junction box 113 should be compatible with combinations of coils of different modes, including two-layer coil (a local transmitting coil/a wearable flexible phased array head coil), a combination of a body coil built in the MM system for transmitting and a wearable flexible phased array head receiving coil, and the single-layer transceiver common phased array coil 15.

In an embodiment, the imaging plug-in device may further include: a to-be-detected object holder located at the magnetic field generated by the magnetic field generation structure, and used to carry the to-be-detected object, wherein the to-be-detected object is a part of the human body.

The to-be-detected object holder is exemplarily described below.

The to-be-detected object holder 16 may be a head holder, a wrist holder, a leg holder and other supporting structures, and may be arranged on the hospital bed of the MM system, and at least part of the imaging plug-in device may be arranged on the hospital bed. At least part of the magnetic resonance receiving coil is arranged on the to-be-detected object, and/or at least part of the magnetic resonance receiving coil is detachably arranged on the to-be-detected object, and/or the magnetic resonance receiving coil includes a flexible phased array coil, and at least part of the flexible phased array coil is arranged in a wearable structure.

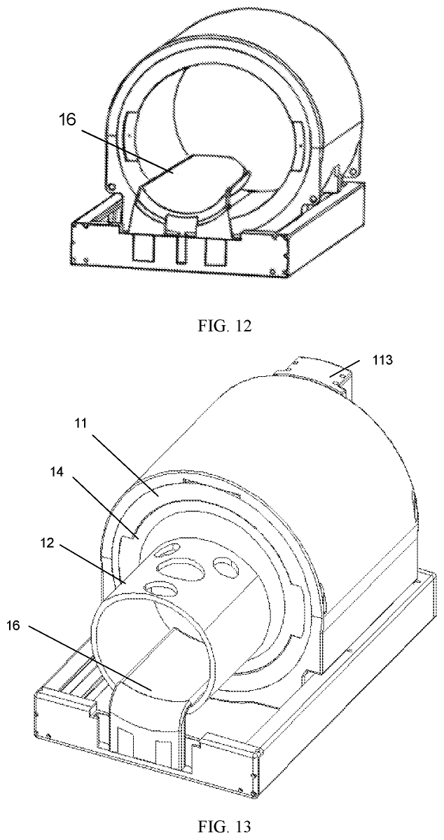

FIG. 12 schematically shows a schematic structural diagram of a to-be-detected object holder according to another embodiment of the present disclosure.

As shown in FIG. 12, the to-be-detected object holder 16 may include a head holder. When the PET detection is required, the head holder may be placed in the PET detection component. In addition, in order to improve the Mill detection effect and detection convenience, the head holder may be connected with the magnetic resonance receiving coil. The receiving coil may not only perform the Mill detection on the to-be-detected object, but also be a portion of a part that fixes a position of the to-be-detected object to ensure an accuracy of a detection result.

In an embodiment, a position of the to-be-detected object holder 16 may be fixed. In addition, in order to improve the convenience of Mill detection and PET detection, the position and posture of the to-be-detected object may be adjusted. For example, at least one of a height, a pitch angle, a heading angle and a roll angle of the to-be-detected object is adjustable. The to-be-detected object holder 16 may include a drive component, such as an electric drive component, a hydraulic drive component, a pneumatic drive component, etc. Through the drive component, the position and posture of the object to-be-detected holder 16 may be changed, such as adjusting at least one of the above-mentioned position and posture parameters.

In an embodiment, the PET detection component 11 further includes a roller-like structure and a moving structure. The PET detector is arranged in the roller-like structure. The moving structure is used to drive the roller-like structure to move along an extension direction of the to-be-detected object holder 16, and the extension direction of the to-be-detected object holder 16 is consistent with an axial direction of the roller.

FIG. 13 schematically shows a structural perspective view of an imaging plug-in device according to embodiments of the present disclosure.

As shown in FIG. 13, the imaging plug-in device may include: the to-be-detected object holder 16, the PET detection component 11, the magnetic resonance phased coil and the signal amplification component.

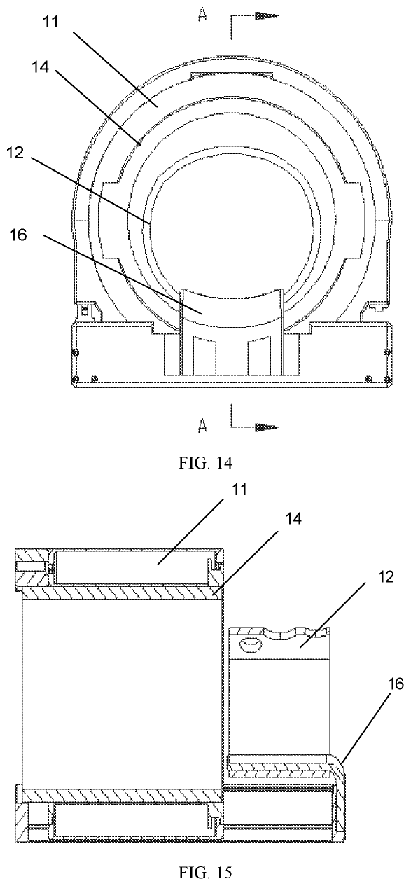

In addition, the imaging plug-in device may further include a mobile structure for driving the PET detection component 11 to move relative to the to-be-detected object holder 16 to align the to-be-detected object or leave the to-be-detected object. The mobile structure includes but is not limited to: a wheel, a guide structure (such as a slide rail, etc.). The magnetic resonance phased coil may further include a local transmitting coil 14. The local transmitting coil 14 may move together with the PET detection component 11. In FIG. 14, the PET detection component 11 is in a state of leaving the to-be-detected object. When the PET detection is required, the PET detection component 11 moves in a direction close to the to-be-detected object holder 16 to align with the to-be-detected object.

FIG. 14 schematically shows a front view of an imaging plug-in device according to embodiments of the present disclosure. FIG. 15 schematically shows A-A sectional view of the imaging plug-in device in FIG. 14.

As shown in FIG. 13, in order to reduce claustrophobia of a user and improve comfort during the PET detection, the magnetic resonance receiving coil 12 may be provided with a plurality of openings to expose at least one of the user's eyes, nose and mouth. It should be noted that the magnetic resonance receiving coil 12 is only shown as an example. In order to facilitate the to-be-detected object to enter the magnetic resonance receiving coil 12, the magnetic resonance receiving coil 12 may be an openable and closable structure or a removable structure, which is not limited here.

The above embodiments are only used to illustrate technical solutions of the present disclosure, and are not intended to limit technical solutions of the present disclosure. Although the present disclosure has been described in detail with reference to the preceding embodiments, those of ordinary skill in the art should understand that the technical solutions described in the preceding embodiments may still be modified, or some or all of the technical features may be equivalently replaced, and such modifications or replacements do not make the essence of the corresponding technical solutions depart from the scope of the technical solutions of embodiments of the present disclosure.

1. An imaging plug-in device, applied to a magnetic resonance imaging system, the magnetic resonance imaging system comprising: a magnetic field generation structure configured to generate a magnetic field; a gradient magnetic field coil configured to form a gradient magnetic field at a to-be-detected object; and a hospital bed configured to carry a human body and at least part of the imaging plug-in device,
wherein the imaging plug-in device comprises:
a to-be-detected object holder located at the magnetic field generated by the magnetic field generation structure, and configured to carry the to-be-detected object, wherein the to-be-detected object is a part of the human body;
a PET (Positron Emission Computed Tomography) detection component configured to detect a PET signal from the to-be-detected object, so as to generate a PET image of the to-be-detected object;
a magnetic resonance phased coil at least comprising a magnetic resonance receiving coil for detecting a magnetic resonance signal from the to-be-detected object, and configured to generate a magnetic resonance image of the to-be-detected object; and
a signal amplification component arranged at a nearest settable place outside a region of interest of the PET detection component, so as to at least improve a signal-to-noise ratio of the magnetic resonance signal;
wherein the PET detection component is movable relative to the to-be-detected object holder to align the to-be-detected object or leave the to-be-detected object.

2. The imaging plug-in device according to claim 1, wherein the magnetic resonance phased coil further comprises a local transmitting coil configured to transmit a radio frequency signal.

3. The imaging plug-in device according to claim 2, wherein the magnetic resonance receiving coil and the local transmitting coil are split, and the local transmitting coil is arranged on a side of the PET detection component close to the to-be-detected object, and moves together with the PET detection component to align or leave the to-be-detected object.

4. The imaging plug-in device according to claim 2, wherein a body coil built in the magnetic resonance imaging system is configured to generate a radio frequency field.

5. The imaging plug-in device according to claim 4, wherein the local transmitting coil is coupled to a local radio frequency transmission interface end of the magnetic resonance imaging system through an external junction box.

6. The imaging plug-in device according to claim 5, wherein the external junction box comprises a radio frequency power distributor.

7. The imaging plug-in device according to claim 6, wherein the radio frequency power distributor is configured to generate two radio frequency signals with a same amplitude and a phase difference of 90 degrees.

8. The imaging plug-in device according to claim 6, wherein an input end of the radio frequency power distributor is coupled to the local radio frequency transmission interface end of the magnetic resonance imaging system, and two output ends of the radio frequency power distributor are respectively coupled to the local transmitting coil.

9. The imaging plug-in device according to claim 5, wherein the signal amplification component is arranged in the external junction box.

10. The imaging plug-in device according to claim 2, wherein the magnetic resonance receiving coil and the local transmitting coil are integrated, and together form a single-layer transceiver common phased array coil.

11. The imaging plug-in device according to claim 10, wherein the single-layer transceiver common phased array coil is arranged on a side of the PET detection component close to the to-be-detected object, and moves together with the PET detection component to align or leave the to-be-detected object.

12. The imaging plug-in device according to claim 10, wherein,
the magnetic resonance phased coil comprises a plurality of channel coils; each channel coil has a respective transmit/receive switch; the transmit/receive switch is configured to switch a working state of the channel coil corresponding to the transmit/receive switch, and the working state comprises: a transmitting state and a receiving state; and
the imaging plug-in device further comprises a multi-channel radio frequency power distributor, wherein each channel of the multi-channel radio frequency power distributor has a one-to-one correspondence with each channel coil.

13. The imaging plug-in device according to claim 12, wherein a phase difference between adjacent channels of an 8-channel coil is 45 degrees, and phases of each channel of the 8-channel coil are not repeated.

14. The imaging plug-in device according to claim 12, wherein the multi-channel radio frequency power distributor comprises a three-level power distributor and a phase shifter.

15. The imaging plug-in device according to claim 1, wherein,
at least part of the magnetic resonance receiving coil is arranged on the to-be-detected object holder; and/or
at least part of the magnetic resonance receiving coil is detachably arranged on the to-be-detected object; and/or
the magnetic resonance receiving coil comprises a flexible phased array coil, and at least part of the flexible phased array coil is arranged in a wearable structure.

16. The imaging plug-in device according to claim 15, wherein the PET detection component further comprises a roller-like structure and a moving structure, wherein a plurality of PET detectors are arranged in the roller-like structure, and the moving structure is configured to drive the roller-like structure to move along an extension direction of the to-be-detected object holder.
